(19) United States
(12) Patent Application Publication
(10) Pub. No.: US 2020/0205990 A1
(43) Pub. Date: Jul. 2, 2020

(54) CALCANEOUS IMPLANT

(71) Applicants: Jacob WAUGH, Irvine, CA (US); Christopher ELKINS, Irvine, CA (US)

(72) Inventors: Jacob WAUGH, Irvine, CA (US); Christopher ELKINS, Irvine, CA (US)

(21) Appl. No.: 16/729,651

(22) Filed: Dec. 30, 2019

Related U.S. Application Data
(60) Provisional application No. 62/785,925, filed on Dec. 28, 2018.

(51) Int. Cl. A61F 2/42; A61F 3/00

(52) U.S. Cl. CPC A61F 2/4202; A61F 3/00; A61F 2310/00005; A61F 2310/00011; A61F 2310/00179; A61F 2002/4217

(57) ABSTRACT

A calcaneal implant for extending the length of a leg of a patient and surgical methods for implanting the implant are described herein.

### B. CROSS-REFERENCE TO RELATED APPLICATIONS

This application claims priority from U.S. Provisional No. 62/785,925, filed Dec. 28, 2018, entitled “Calcaneus Implant,” the entirety of which is hereby incorporated by reference.

### C. GOVERNMENT INTERESTS

Not applicable

### D. PARTIES TO A JOINT RESEARCH AGREEMENT

Not applicable

### E. INCORPORATION OF MATERIAL ON COMPACT DISC

Not applicable

### F. BACKGROUND

Leg length discrepancy (LLD), or anisomelia, is an orthopedic condition in which two limbs are of unequal lengths. LLD can appear in childhood or as a result of injury such as bone fractures that cause damage to the growth plate, or illness, such as bone infections or bone tumors, or LLD can be congenital or developmental appearing at birth and increasing progressively as the child grows. In some cases, an illness or injury that causes damage to growth plate can also lead to the development of a limb length discrepancy, or short leg syndrome, later in life. While differences in arm lengths often require no treatment, a leg length discrepancy can cause a noticeable limp or make it difficult for patients to perform normal activities. When the difference measures more than 1.5 to 2.0 cm (⅝ in) in length, physicians often require treatment or surgical intervention. LLD patients experience numerous often painful symptoms including pelvic shift, low back pain (LBP), idiopathic scoliosis, iliotibial band syndrome, foot pronation, and stress fractures in a lower extremity. Current surgical treatments are limited to limb stretching surgery, which requires bones in the limbs of patients to be broken and stretched repeatedly using implanted scaffolding, an extremely painful and time-consuming procedure.

### G. SUMMARY OF THE INVENTION

Various embodiments of the invention are directed to implantable devices including a body having a generally half spherical shape, concave inner surface formed from upward extensions of lateral surfaces of the body and an upward extension of the rear surface body, and a distal extension extending from one surface of the body, and a posterior thickening below the concave inner surface extending away from the circumferential surface of the body. In some embodiments, the extensions of the lateral surfaces of the body taper to form the distal extension. In some embodiments, the distal extension terminates in a pointed, rounded, or arched shape. In certain embodiments, the implantable device may include a pad on a surface of the hollow inner surface, and in some embodiments, the pad may be composed of a filler material. In various embodiments, the implantable device may further include one or more anchors or fasteners. The body of embodiments may be composed of implantable plastic, implantable polymer, implantable metal, implantable ceramic, or combinations thereof.

Other embodiments are directed to a method for lengthening a limb including the steps of fixedly attaching to a calcaneus bone, an implantable device including a body having a generally half spherical shape, concave inner surface formed from upward extensions of lateral surfaces of the body and an upward extension of the rear surface body, and a distal extension extending from one surface of the body, and a posterior thickening below the concave inner surface extending away from the circumferential surface of the body. In some embodiments, the method may include the step of connecting the implantable device to surrounding soft tissue, metatarsals, and combinations thereof using sutures, anchors, adhesives or combinations thereof. In some embodiments the method may further include the step of anchoring the implantable device to a calcaneus, calcaneal tuberosity, or combinations or portions thereof using anchors, fasteners, screws, adhesives, or combinations thereof. In some embodiments, the extensions of the lateral surfaces of the body taper to form the distal extension. In some embodiments, the distal extension terminates in a pointed, rounded, or arched shape. In certain embodiments, the implantable device may include a pad on a surface of the hollow inner surface, and in some embodiments, the pad may be composed of a filler material. In various embodiments, the implantable device may further include one or more anchors or fasteners. The body of embodiments may be composed of implantable plastic, implantable polymer, implantable metal, implantable ceramic, or combinations thereof.

### H. DESCRIPTION OF THE DRAWINGS

Examples of the specific embodiments are illustrated in the accompanying drawings. While the invention will be described in conjunction with these specific embodiments, it will be understood that it is not intended to limit the invention to such specific embodiments. On the contrary, it is intended to cover alternatives, modifications, and equivalents as may be included within the spirit and scope of the invention. In the following description, numerous specific details are set forth in order to provide a thorough understanding of the present invention. The present invention may be practiced without some or all of these specific details. In other instances, well known process operations have not been described in details so as to not unnecessarily obscure the present invention.

FIG. 1 is an illustration of a human foot showing the bones and joints associated with the heel.

FIG. 2A is a detailed illustration of an anterior view of the calcaneus bone.

FIG. 2B is a detailed illustration of a posterior view of the calcaneus bone.

FIG. 2C is a detailed illustration of a lateral view of the calcaneus bone.

FIG. 3 is an illustration of an implantable device of embodiments attached to a calcaneus bone.

FIG. 4A is a lateral view of an implantable device of embodiments.

FIG. 4B is a posterior view of an implantable device of embodiments.

FIG. 4C is an anterosuperior view of an implantable device of embodiments.

FIG. 4D is an inferior view of an implantable device of embodiments.

### I. DETAILED DESCRIPTION

Various aspects now will be described more fully hereinafter. Such aspects may, however, be embodied in many different forms and should not be construed as limited to the embodiments set forth herein; rather, these embodiments are provided so that this disclosure will be thorough and complete, and will fully convey its scope to those skilled in the art.

Where a range of values is provided, it is intended that each intervening value between the upper and lower limit of that range and any other stated or intervening value in that stated range is encompassed within the disclosure. For example, if a range of 1 mm to 8 mm is stated, 2 mm, 3 mm, 4 mm, 5 mm, 6 mm, and 7 mm are also intended to be explicitly disclosed, as well as the range of values greater than or equal to 1 mm and the range of values less than or equal to 8 mm.

All percentages, parts and ratios are based upon the total weight of the topical compositions and all measurements made are at about 25° C., unless otherwise specified.

The singular forms “a,” “an,” and “the” include plural referents unless the context clearly dictates otherwise. Thus, for example, reference to a “polymer” includes a single polymer as well as two or more of the same or different polymers; reference to an “excipient” includes a single excipient as well as two or more of the same or different excipients, and the like.

The word “about” when immediately preceding a numerical value means a range of plus or minus 10% of that value, e.g, “about 50” means 45 to 55, “about 25,000” means 22,500 to 27,500, etc, unless the context of the disclosure indicates otherwise, or is inconsistent with such an interpretation. For example, in a list of numerical values such as “about 49, about 50, about 55, “about 50” means a range extending to less than half the interval(s) between the preceding and subsequent values, e.g, more than 49.5 to less than 52.5. Furthermore, the phrases “less than about” a value or “greater than about” a value should be understood in view of the definition of the term “about” provided herein.

The transitional term “comprising,” which is synonymous with “including,” “containing,” or “characterized by,” is inclusive or open-ended and does not exclude additional, unrecited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention. In embodiments or claims where the term comprising is used as the transition phrase, such embodiments can also be envisioned with replacement of the term “comprising” with the terms “consisting of” or “consisting essentially of.”

The term “patient” and “subject” are interchangeable and may be taken to mean any living organism which may be treated with compounds of the present invention. As such, the terms “patient” and “subject” may include, but is not limited to, any non-human mammal, primate or human. In some embodiments, the “patient” or “subject” is a mammal, such as mice, rats, other rodents, rabbits, dogs, cats, swine, cattle, sheep, horses, primates, or humans. In some embodiments, the patient or subject is an adult, child or infant. In some embodiments, the patient or subject is a human.

The term “treating” is used herein, for instance, in reference to methods of treating a skin disorder or a systemic condition, and generally includes the administration of a compound or composition which reduces the frequency of, or delays the onset of, symptoms of a medical condition or enhance the texture, appearance, color, sensation, or hydration of the intended tissue treatment area of the tissue surface in a subject relative to a subject not receiving the compound or composition. This can include reversing, reducing, or arresting the symptoms, clinical signs, and underlying pathology of a condition in a manner to improve or stabilize a subject's condition.

This description of preferred embodiments is intended to be read in connection with the accompanying drawings, which are to be considered part of the entire written description. The drawing figures are not necessarily to scale and certain features may be shown exaggerated in scale or in somewhat schematic form in the interest of clarity and conciseness. I n the description, relative terms such as “horizontal,” “vertical,” “up,” “down,” “top” and “bottom” as well as derivatives thereof (e.g., “horizontally,” “downwardly,” “upwardly,” etc.) should be construed to refer to the orientation as then described or as shown in the drawing figure under discussion. These relative terms are for convenience of description and normally are not intended to require a particular orientation. Terms including “inwardly” versus “outwardly,” “longitudinal” versus “lateral” and the like are to be interpreted relative to one another or relative to an axis of elongation, or an axis or center of rotation, as appropriate. Terms concerning attachments, coupling and the like, such as “connected” and “interconnected,” refer to a relationship wherein structures are secured or attached to one another either directly or indirectly through intervening structures, as well as both movable or rigid attachments or relationships, unless expressly described otherwise. When only a single machine is illustrated, the term “machine” shall also be taken to include any collection of machines that individually or jointly execute a set (or multiple sets) of instructions to perform any one or more of the methodologies discussed herein. The term “operatively connected” is such an attachment, coupling or connection that allows the pertinent structures to operate as intended by virtue of that relationship. In the claims, means-plus-function clauses, if used, are intended to cover the structures described, suggested, or rendered obvious by the written description or drawings for performing the recited function, including not only structural equivalents but also equivalent structures.

By hereby reserving the right to proviso out or exclude any individual members of any such group, including any sub-ranges or combinations of sub-ranges within the group, that can be claimed according to a range or in any similar manner, less than the full measure of this disclosure can be claimed for any reason. Further, by hereby reserving the right to proviso out or exclude any individual substituents, analogs, compounds, ligands, structures, or groups thereof, or any members of a claimed group, less than the full measure of this disclosure can be claimed for any reason. Throughout this disclosure, various patents, patent applications and publications are referenced. The disclosures of these patents, patent applications and publications in their entireties are incorporated into this disclosure by reference in order to more fully describe the state of the art as known to those skilled therein as of the date of this disclosure. This disclosure will govern in the instance that there is any inconsistency between the patents, patent applications and publications cited and this disclosure.

For convenience, certain terms employed in the specification, examples and claims are collected here. Unless defined otherwise, all technical and scientific terms used in this disclosure have the same meanings as commonly understood by one of ordinary skill in the art to which this disclosure belongs.

Leg length discrepancy (LLD), or anisomelia, is an orthopedic condition in which two limbs are of unequal lengths. LLD can appear in childhood or as a result of injury such as bone fractures that cause damage to the growth plate, or illness, such as bone infections or bone tumors, or LLD can be congenital or developmental appearing at birth and increasing progressively as the child grows. In some cases, an illness or injury that causes damage to growth plate can also lead to the development of a limb length discrepancy, or short leg syndrome, later in life. While differences in arm lengths often require no treatment, a leg length discrepancy can cause a noticeable limp or make it difficult for patients to perform normal activities. When the difference measures more than 1.5 to 2.0 cm (⅝ in) in length, physicians often require treatment or surgical intervention. LLD patients experience numerous often painful symptoms including pelvic shift, low back pain (LBP), idiopathic scoliosis, iliotibial band syndrome, foot pronation, and stress fractures in a lower extremity. Current surgical treatments are limited to limb stretching surgery, which requires bones in the limbs of patients to be broken and stretched repeatedly using implanted scaffolding, an extremely painful and time-consuming procedure.

Various embodiments of the invention are directed implants designed and configured to be surgically attached to the calcaneus bone of a human foot and methods for implanting such implants in a patient. The implants may increase the length of the patient's leg by up to about 2 inches, without breaking bones, e.g. fibula, tibia, or femur, repeated surgeries, or substantial rehabilitation or gait training. The implants of such embodiments may provide a viable treatment for leg length discrepancy and relief from symptoms including, pelvic shift, low back pain (LBP), idiopathic scoliosis, iliotibial band syndrome, foot pronation, stress fractures lower extremity, and the like and combinations thereof.

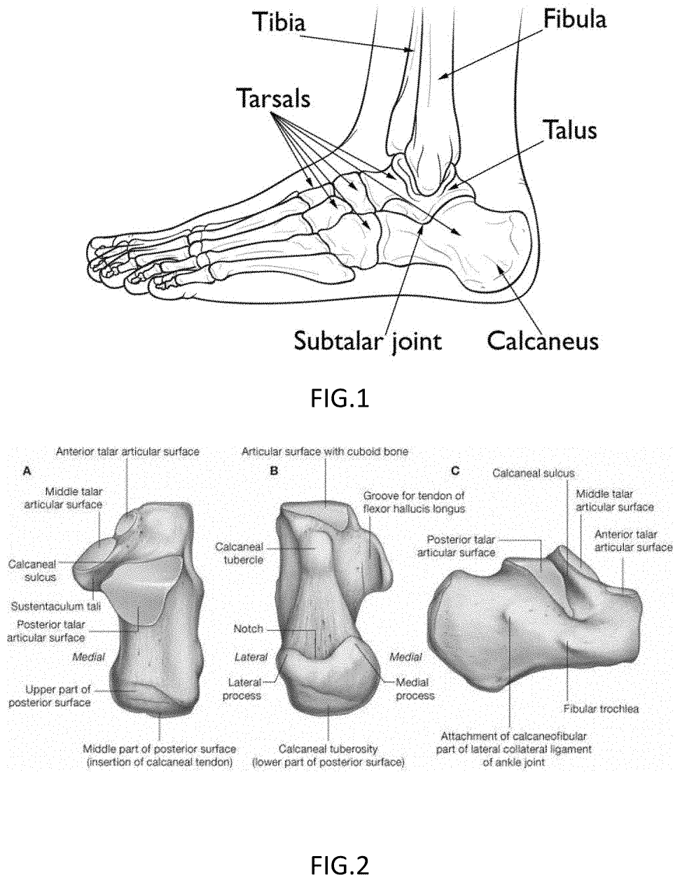

FIG. 1A and 1B provides diagrams of the foot showing the anatomy of the bones of the foot. As illustrated in FIG. 2, the calcaneus implants 20 of various embodiments may be shaped and sized to attach to and extend the lower part of the posterior surface of the calcaneus bone, i.e. the calcaneal tuberosity (see FIG. 1B), lengthening the posterior surface of the calcaneus by up to about 2 inches or 2.5 inches and increasing the length of the leg and height of the patient by this length. The implant 20 of such embodiments may be attached to the calcaneus by anchors, sutures, or adhesives. In some embodiments, the implant 20 may be attached on one leg of the patient to treat LLD, and in other embodiments, the implant 20 may be attached to both legs of the patient to increase the overall height of the patient by up to about 2.5 inches.

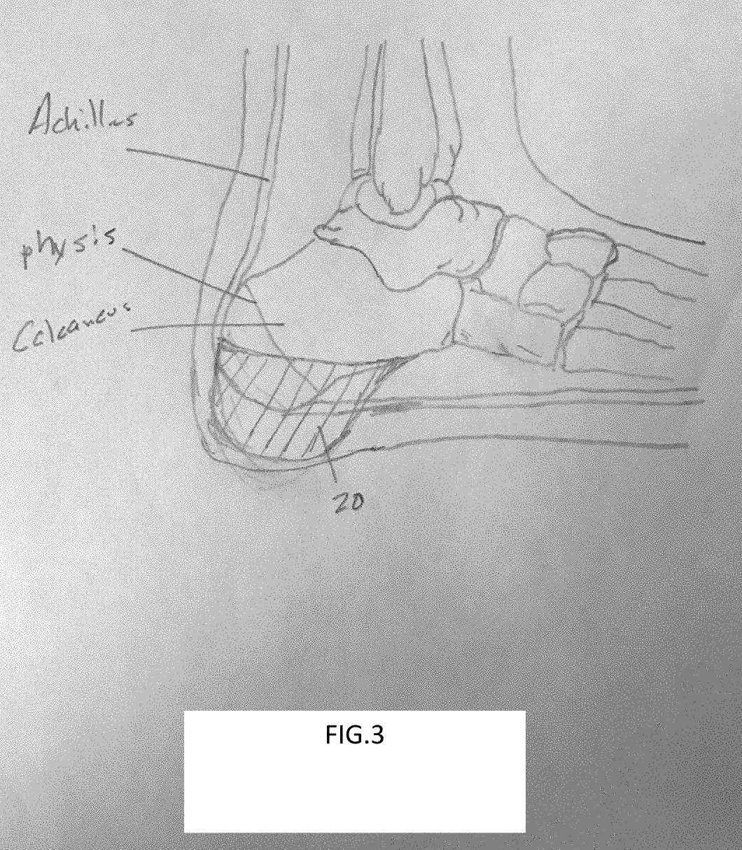

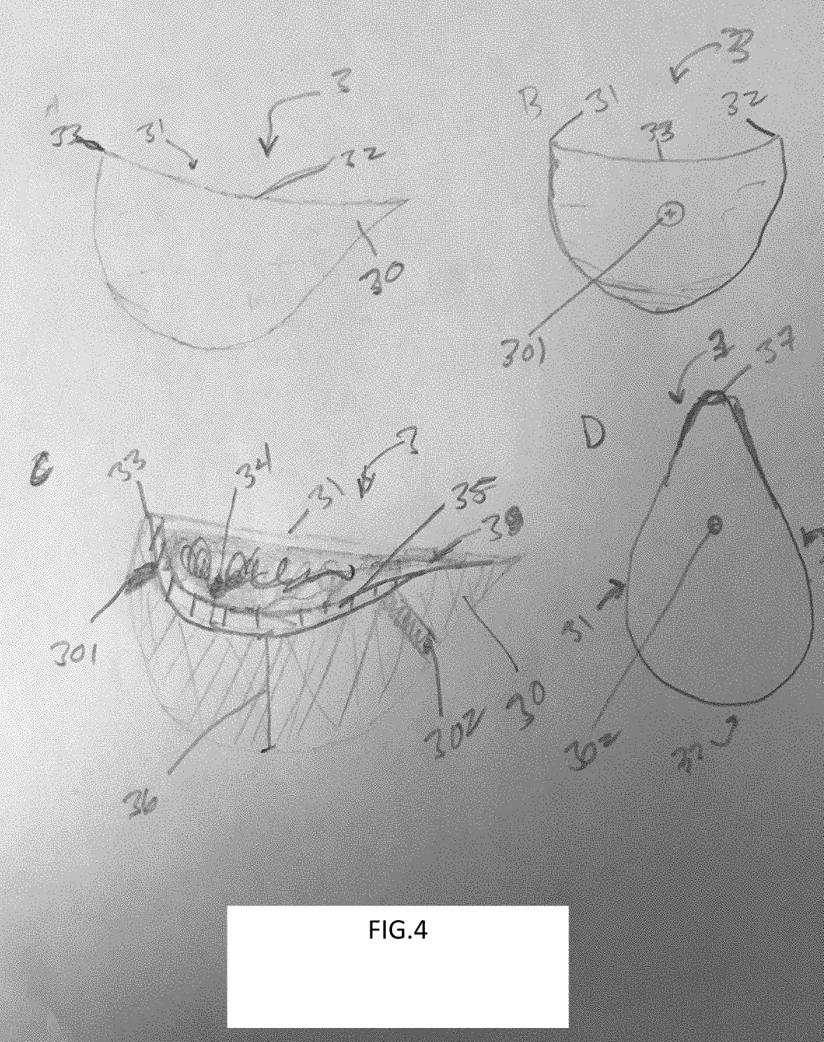

The calcaneus implant may include a body 3 having a generally half spherical shape, FIG. 3A,3B, that mimics the general shape of the calcaneus tuberosity, and in some embodiments includes a distal extension 30 that allows the implant to meet the upper part of the posterior surface of the calcaneus to below the fibular trochlea (see FIG. 1B). At least one portion of the body 3 may be thickened 36 to extend the posterior surface of the calcaneus, increasing the length of the calcaneus and increasing the overall length of the leg of the patient by at least about 2 inches or 2.5 inches, see FIG. 3C.

Lateral surfaces 31, 32 of the calcaneus body 3 may extend upward meeting the medial and lateral processes of the calcaneal tuberosity (see FIG. 1B), and a rear surface 33 of the calcaneus body 3 may extend upward meeting the notch (see FIG. 1B). These extensions of the lateral surfaces create a hollow inner surface 34 as illustrated FIG. 3C that provides a surface that meets substantial portions of the calcaneus tuberosity when the body 3 is operably associated with the calcaneus. In some embodiments, the calcaneus implant may further include a pad 35 operably attached to a substantial portion of the inner surface 34 of the body 3 that increases the surface area of the association between the body 3 and the calcaneus tuberosity and provide padding for the calcaneus.

The distal extension 30 may taper from the lateral surfaces 31,33 to a point 37 designed and arranged to meet the upper part of the posterior surface of the calcaneus, see FIG. 3D. The point 37, may be pointed or rounded or arched. The distal extension 30 may include a recess 38 to facilitate the association between the body 3 and the calcaneus. In some embodiments, the recess 38 may include padding or the pad 35 of the hollow inner surface 34 may be extended into the recess 38 to increase the surface area of the association between the body 3 and the calcaneus tuberosity and provide padding for the calcaneus.

The body 3 can be attached to the calcaneus by any means. For example, in some embodiments, the implant may include one or more anchors that facilitate attachment of the implant to calcaneus. In certain embodiments, holes 301 may be positioned on the rear of the body 3 to allow of one or two axial screws, or other anchors, to be directed into the posterior of tuberosity toward the dorsal portion of the anterior process, and in some embodiments, holes 302 may be positioned on the lower portion of the body 3 to allow for one or two axial screws, or other anchors, to be directed into the upper part of the posterior surface of the calcaneus toward the he dorsal portion of the posterior process. In other embodiments, the body 3 may be attached to the calcaneus using an adhesive, and in still other embodiments, staples, sutures, or other known surgical means for attaching the body 3 to the calcaneus. In yet other embodiments, a combination of attachment means can be used to attach the body 3 to the calcaneus.

The implants of various embodiments described above can be composed of various materials, including implantable plastic, polymer, metal, or combination thereof suitable for implanting in a human or mammal. Such metals include, but are not limited to titanium, titanium alloys, stainless steel, stainless steel alloys, cobalt-chromium-molybdenum alloy, tantalum, tantalum alloy, nitinol, ceramics, and combinations thereof. Implantable grade synthetic plastic can be thermoplastic or thermoset materials, and include, for example, PEEK (polyether ether ketone), PEEK-Optima HA enhanced polymer, PEKK (polyether ketone ketone), polyethylene, ultra high molecular weight polyethylene, polyphenylsulfone, polysulfone, polythermide, acetal copolymer, woven or solid polyester, implantable grade lennite UHME-PE, and the like and combinations thereof. In some embodiments, the body 3 may be solid, and in other embodiments, the body 3 may have a series of holes to reduce the weight of body 3. In some embodiments, the body may be 3D printed to obtain mesh like or porous structure to allow bone ingrowth (this pertains mostly to titanium but may on the future include PEEK polymer). In some embodiments, the body may be surface treated through mechanical means such as bead blasting or chemically etched or laser etched or electrically etched to produce osteoconductive surfaces.

Further embodiments are directed to surgical methods for implanting the calcaneus implant described above. For example, in some embodiments, such methods may include the steps of making an incision on a medial or lateral surface of the heel of the patient's foot. The initial incision can be approximately 15 mm deep and can be deepened via sharp and blunt dissection to the subdermal layer. The calcaneus can be visualized and separated from any surrounding tissue, exposing the area from lower part of the posterior surface of the calcaneal tuberosity to the upper part of the posterior surface of the calcaneal tuberosity. In some embodiments, the incision can be extended in one or both directions to include the entire desired area of implant. The method may further include introducing the implant into the incision site. The implant can be placed sterilely into the implant site, taking care not to entrap or damage any neurovascular structures or tendon and ligament attachment sites. The implant can then be secured in place by the use of sutures, anchors, or adhesives connecting the implant to the surrounding soft tissue and/or metatarsals. In some embodiments, implant can be anchored to the calcaneus, the calcaneal tuberosity, and other portions of calcaneus.

In some embodiments, the method may further include the step of filling the implant, or junctions between the implant and the calcaneus, with the filler. The implant can then be monitored to ensure that the correct level of filler has been used, and that there has been no shift in the position of the implant. The filler may be introduced through the ports of the implant. In some embodiments, the implant may be pre-filled or solid and in some embodiments, the filler may be the pad discussed above. Finally, the method may include closing the incision.

The surgery associated the method described above can take place in a hospital operating room or in a doctor's office, or it can be done on an outpatient basis. The conditions and technique of the surgery will ideally be sterile and aseptic.

1. An implantable device comprising:
a body having a generally half spherical shape, concave inner surface formed from upward extensions of lateral surfaces of the body and an upward extension of the rear surface body, and a distal extension extending from one surface of the body, and
a posterior thickening below the concave inner surface extending away from the circumferential surface of the body.

2. The implantable device of claim 1, wherein the extensions of the lateral surfaces of the body taper to form the distal extension.

3. The implantable device of claim 2, wherein the distal extension terminates in a pointed, rounded, or arched shape.

4. The implantable device of claim 1, further comprising a pad on a surface of the hollow inner surface.

5. The implantable device of claim 4, wherein the pad is composed of a filler material.

6. The implantable device of claim 1, further comprising one or more anchors or fasteners.

7. The implantable device of claim 1, wherein the body is composed of implantable plastic, implantable polymer, implantable metal, implantable ceramic, or combination thereof.

8. A method for lengthening a limb comprising:
fixedly attaching to a calcaneus bone, an implantable device comprising:
a body having a generally half spherical shape, concave inner surface formed from upward extensions of lateral surfaces of the body and an upward extension of the rear surface body, and a distal extension extending from one surface of the body, and
a posterior thickening below the concave inner surface extending away from the circumferential surface of the body.

9. The method of claim 8, further comprising connecting the implantable device to surrounding soft tissue, metatarsals, and combinations thereof using sutures, anchors, adhesives or combinations thereof.

10. The method of claim 8, further comprising anchoring the implantable device to a calcaneus, calcaneal tuberosity, or combinations or portions thereof using anchors, fasteners, screws, adhesives, or combinations thereof.

11. The method of claim 8, wherein the extensions of the lateral surfaces of the body taper to form the distal extension.

12. The method of claim 8, wherein the distal extension terminates in a pointed, rounded, or arched shape.

13. The method of claim 8, further comprising a pad on a surface of the hollow inner surface.

14. The method of claim 13, wherein the pad is composed of a filler material.

15. The method of claim 8, wherein the body is composed of implantable plastic, implantable polymer, implantable metal, implantable ceramic, or combination thereof.
